(19) United States
(12) Patent Application Publication
(10) Pub. No.: US 2018/0273582 A1
(43) Pub. Date: Sep. 27, 2018

(54) SMALL MOLECULE AFFINITY MEMBRANE PURIFICATION SYSTEMS AND USES THEREOF

(71) Applicant: UNIVERSITY OF NOTRE DAME, Notre Dame, IN (US)

(72) Inventors: Basar BILGICER, Notre Dame, IN (US); Nur MUSTAFAOGLU, Notre Dame, IN (US); Tanyel KIZILTEPE BILGICER, Notre Dame, IN (US)

(21) Appl. No.: 15/774,979

(22) PCT Filed: Nov. 8, 2016
(86) PCT No.: PCT/US2016/061011

§ 371 Date: May 9, 2018

Related U.S. Application Data
(60) Provisional application No. 62/252,628, filed on Nov. 9, 2015.

(51) Int. Cl. C07K 1/22; C07K 16/28; B01J 20/26; B01J 20/28; B01J 20/288; B01J 20/32; B01D 15/38; B01D 71/10

(52) U.S. Cl. CPC C07K 1/22; C07K 16/2887; B01J 20/265; B01J 20/2805; B01J 20/288; C07K 2317/24; B01J 20/3212; B01J 20/3217; B01J 20/3246; B01D 15/3828; B01D 71/10; B01J 20/321

(57) ABSTRACT

Disclosed are purification systems and methods for providing purified preparations of antibodies from a fluid, particularly a biological fluid comprising or suspected to contain antibody (e.g., blood, serum, plasma, ascites fluid). Reusable and stable synthetic purification columns comprising membranes of a suitable separation matrix material, such as a nylon membrane or regenerated cellulose membrane, having conjugated thereto a small molecule capture ligand, such as a short peptide or protein capable of acting as a ligand for a particular antibody of interest, such as a peptide having a sequence with binding affinity for a nucleotide binding site (NBS) of a selected antibody of interest, are also provided. Methods of preparing the purification columns are also disclosed. Methods for preparing high yield and high purity therapeutic antibody preparations, such as anti-cancer therapeutics, from a biological fluid, are also presented.

### CROSS-REFERENCE TO RELATED APPLICATIONS

The present application claims priority to provisional patent application 62/252,628, filed Nov. 9, 2015. The contents of provisional application 62/252,628 is incorporated herein in its entirety.

### STATEMENT REGARDING FEDERALLY SPONSORED RESEARCH OR DEVELOPMENT

None

### FIELD OF THE INVENTION

The present invention relates to the field of affinity membrane purification systems, and uses of such systems in purified antibody preparation.

### BACKGROUND OF THE INVENTION

Antibodies have extraordinary specificity and affinity to antigens, which in turn makes them important candidates to be used in numerous applications including detection, diagnosis, and therapy. Therapeutic antibodies have continued to be evaluated extensively for treatment of many diseases including cancer and autoimmune diseases. Even though antibody therapies are very efficacious for patients, monoclonal antibody-based treatments are expensive; therefore many patients cannot afford these treatments.

Antibodies are employed in a vast array of applications, from diagnostic to therapeutic, while new applications for their implementation are continuously being explored [1-6]. lgG, a divalent antibody having two antigen binding sites, is the most abundant antibody isotype in the human body. It has been of particular interest for research in pharmaceutical industry since FDA approval of Orthoclone in 1986 [7, 8]. Currently, more than 30 monoclonal antibodies and two antibody-drug conjugates (ADC) have been approved for use in many instances, including cancer [1, 9]. Moreover, hundreds of monoclonal antibodies and about 30 new ADC are currently undergoing clinical evaluation [9, 10]. Hence, antibody based therapies will be a major source of new therapeutic approaches, but will require the high purification of antibodies.

The downstream production costs of purifying antibodies to render them suitable as a treatment render them very expensive, and hence a deterrent to many patients. A classical antibody purification process requires four to five independent downstream process steps including primary recovery steps, adsorption of antibodies, polishing steps and finally buffer exchange and concentration steps [11-14]. More than 50-80% of the total cost of protein production is due to these downstream steps [15, 16]. Therefore, antibody purification is still a challenging problem in biomedical applications. Fast, efficient and cheap methods are needed to purify antibodies for supplying the industrial necessities. In this study, we report a novel small-molecule based affinity chromatography method for antibody purification via nucleotide binding site (NBS).

Affinity chromatography with high specificity properties due to the strong interaction between the ligand and the proteins of interest is the leading method in industry that has been used for antibody purification [16, 17]. Protein A and Protein G are by far extensively used ligands for monoclonal antibody purification from crude extracts. Thus, protein A (or G) affinity chromatography is the current industrial standard for antibody purification processes [14, 18, 19].

A major contributor to the cost of downstream production process in purifying antibody is the usage of Protein A (or G) affinity columns for purification of antibodies. These columns are expensive and have short life cycles with several obstacles that prevent them from being used repeatedly.

In this chromatography method, protein A (or G) binds to the antibody Fe domains to remove contaminant such as proteins, DNA, and other impurities from the cell culture process. Although this technique is reported to yield>90% antibody purity [14, 20] there are several problems associated with its use. Natural affinity ligands are produced by recombinant bacterial systems. Their isolation and purification from microbial extract are difficult and require accurate analytical tests to ensure the absence of toxic contaminant; hence it causes significantly high production cost. Large proteins such Protein A (42 kDa) and Protein G (˜65 kDa) have been affected with small environmental changes [21, 22]. The proteins may denature, loss their tertiary structure and binding affinity over time, which causes several problems in antibody purification procedure such as contamination of purified antibodies due to leaching of Protein NG fragments, and inability to purify misfolded and/or denatured antibodies [23-28]. Elution from Protein A affinity adsorbents are effective under conditions of low pH which involves possibility of denaturation of eluted antibodies as well as aggregation problems [29-31]. Additionally, the standard non-oriented methods for immobilization of Protein A (or G) to solid supports can result in a significant loss of binding activity due to steric constraints, yielding reduced column capacity [32].

Alternative to Protein A, several approaches have been taken in the quest for simple, selective and stabile ligands [33, 34]. Generally, the simpler the ligands, the more stable it is to harsh chemical procedures for elution and cleaning. Therefore, extensive efforts have been spent examining small molecules such as chlorizene dyes [16], histidine [35] thiophilic compounds [36] and small peptides [31] that show promising facilities (high binding capacity and excellent chemical stability) to be used as an alternative to Protein A (or G) with varying selectivity and complexity. On the other hand, simplicity comes with a lower degree of selectivity. Thus, this is still a crucial problem in affinity chromatography that has yet to be resolved.

Membrane chromatography systems possess several advantages over affinity resin-based chromatography. The membrane provides well-controlled macro-porous polymeric stationary phases which leads to a lower pressure drop and higher flow rate [17]. Membrane based chromatography generally can be distinguished from resin-based chromatography through its interaction between a solute and a matrix (immobilized ligand) and does not take place in the dead-ended pores of a particle, but mainly in the through pores of a membrane. As a result of convective flow of the solution through the pores, the mass transfer resistance is reduced and rapid processing, which improves the adsorption, washing, elution and regeneration steps, can be achieved [17, 37]. The binding efficiency is generally independent of the feed flow-rate over a wide range and therefore very high flow-rate may be used [38]. Therefore, a larger sample size can be processed in a relatively short time with high recovery of activity [17]. Additionally, easy packing and scale up facilities of membranes makes them more preferable in antibody purification systems [39, 40]. Production of membranes is generally easy and inexpensively, thereby they can be replaced easily after ceasing their function properly, which eliminates the requirement for cleaning and equipment revalidation [38]. All of these features and advantages over resin-based systems make membranes a good candidate to be used in affinity chromatography systems.

While these and other advantages for using a membrane verses a resin have been observed, a system for utilizing other than a resin-based system, and/or alternatives to the use of relatively large and costly proteins in the purification technique, such as protein A, have not been proposed.

The clinical and medical arts remain in need of tools and more economical techniques for purification of antibodies, while maintaining high quality antibody preparation.

### SUMMARY OF THE INVENTION

In a general and overall sense, the present disclosure provides small molecule affinity purification systems, and purified antibody preparations prepared using these systems.

In one aspect, the system comprises an affinity membrane chromatography technique that includes a small molecule capture ligand affixed to a separation matrix.

In some embodiments, the system may provide for the purification of monoclonal and polyclonal antibodies from a biological fluid. By way of example, such biological fluids may comprise cell culture media in which a cell has been cultured, blood, serum, plasma, ascites fluid, urine, or other biological fluid that may include an antibody or other molecule of interest having at least some binding affinity for the small molecule capture ligand.

According to some embodiments, the method may be used for the purification of an antibody of interest. In other embodiments, the method for purifying an antibody of interest from a fluid (such as from a biological fluid) comprises providing a separation column comprising a separation matrix, said separation matrix having affixed thereto a small molecule capture ligand having binding affinity for the antibody of interest, providing a fluid to the separation column, wherein said small molecule capture ligand will bind to the antibody of interest that may be present in the fluid and that has a sufficient binding affinity for the small molecule capture ligand; eluting the separation matrix (such as a separation matrix provided in the form of a separation column) with an elution fluid, selecting elution fractions containing the antibody of interest for collection, and purifying the antibody of interest from the selected fractions.

In some embodiments, the separation matrix comprises a membrane or series of membranes. The membranes may comprise a regenerated cellulose membrane or other material, such as a nylon membrane. By way of example, a regenerated cellulose membrane may comprise polyethersulfone or polyvinylidene fluoride. The separation matrix may comprise other types of membranes, including one or more membranes as components of a separation matrix or separation column. For example, the separation matrix may comprise a monolithic column, or other column configuration. Among other advantages, the separation matrix and membranes comprise materials that are highly resistant to degradation, such as degradation associated with particular types of buffers and elution fluids, as well as remain stable and effective for separation across a wide ranges of pH conditions.

The separation matrices and membranes also provide high predictability in separation efficiency, purity and yield, and provides a separation technique that accommodates a highly controlled methodology, accommodating relatively high flow rates of buffer through the matrix. Higher flow rates permits a more rapid separation of antibody from a test sample, such as a biological fluid. The separation matrix, membranes, and separation columns comprising them, are shown to provide sharp peaks of isolated antibody, rendering the method an effective and efficient tool for producing high purity antibody products at yields of up to 80% or greater (such as 90%, 95% and even 98%). The materials and methods provided herein will provide an at least 60% yield of a desired antibody.

By way of example, the antibody of interest may comprise a monoclonal antibody or polyclonal antibody, or a native antibody or a recombinant antibody, such as a chimeric antibody. By way of example, the chimeric antibody may comprise a humanized monoclonal antibody. In particular, the antibody being purified may comprise Rituximab.

The small molecule capture ligand may be further described as a peptide having an amino acid sequence that demonstrates binding affinity for a nucleotide binding site (NBS). While the NBS present on an antibody has no known function, this region has been identified as providing a “pocket” within which a suitable small molecule affinity ligand may bind, and thus capture, an antibody. This system is used in the present methods and compositions, having identified the NBS as a target around which the improved antibody purification techniques are fashioned. The NBS region of an antibody is a highly conserved region among antibodies generally. Small molecule affinity ligands that target this NBS provide tools in a purification system that achieves high purity and high yield of virtually any antibody of interest. By way of example, the small molecule capture ligand is a peptide having an indole ring. By way of further example, the small molecule capture ligand may comprise tryptamine or other molecule demonstrating the same or similar ligand-binding properties for an NBS region of an antibody, and having an indole-ring structure.

In some embodiments, the small molecule capture ligand may comprises a peptide having a sequence that possesses sufficient binding affinity for a variable domain region of an FAB region of an antibody of interest to be purified.

The NBS may be further defined as comprising an amino acid sequence of four amino acids, these four amino acids being three tyrosine residues and one tryptophan residue, these amino acid residues relating to two tyrosine residues located on the variable region of an antibody light chain (VL) (Tyr42 and Try103) and one tyrosine (Try103) and one tryptophan (Trp118) residue located on the variable region of an antibody heavy chain (VH). The NBS functions as a capture “pocket” on an antibody of interest, serving to allow the small molecule capture ligand affixed to a separation matrix/membrane to capture the antibody onto a separation column.

According to one description, the small molecule capture ligand may be described as comprising a peptide having an indole ring structure and an amino acid sequence that demonstrates moderate binding affinity at a pH of about 7, for a highly conserved region of an antibody of interest. This highly conserved region of the antibody is the NBS.

While virtually any fluid may be screened to discern the presence of antibody, it is envisioned that the fluid may comprise a cell culture media in which cells have been cultured, or any number of different biological fluids or residual biological fluid. As used in the description here, a residual biological fluid may comprise a fluid that is a by-product or discarded fraction or eluent from a laboratory or clinical processing or procedure, in which residual antibody may be harvested. By way of example, a biological fluid may comprise an ascites fluid, blood, serum, or plasma.

In particular embodiments, the antibody of interest will comprise a therapeutic antibody, such as an antibody that may be used as an anti-cancer therapeutic agent.

In yet another aspect, a reusable antibody purification synthetic substrate is provided. In some embodiments, the solid substrate comprising a regenerated cellulose membrane, and a small molecule affinity ligand conjugated to said substrate, wherein the small molecule affinity ligand has an indole structure and binding affinity for a small highly conserved sequence of a variable domain in mammalian antibody. In particular embodiments, the regenerated cellulose membrane is functionalized to include carboxyl groups, thus providing a carboxylated membrane, and then activated. In one example, the small molecule affinity ligand is tryptamine, or other small peptide having binding affinity characteristics and size similar to tryptamine. In some embodiments, the regenerated cellulose membrane comprises polyethersulfone or polyvinylidene fluoride. In a particular embodiment, the reusable antibody purification synthetic substrate is an m-NBS^{Tryptamine} affinity column.

In yet another aspect, the invention provides an antibody purification kit comprising the reusable synthetic substrate described herein, together with an insert providing directions on the use of the substrate according to the present methods to purify an antibody of interest.

In some aspects, the small binding ligand described here may be further described as utilizing a nucleotide-binding site (NBS) that is located on the variable domain of aFab region of nearly all antibodies (i.e., the region is highly conserved among mammalian antibodies). This NBI may be used to facilitate the capture of virtually any antibody on the membrane affinity column disclosed here.

The solid substrates may be further described as comprising a material, such as a separation matrix other than resin, and particularly as comprising regenerated cellulose membranes that are essentially free of resin, to provide a matrix. The nature of the disclosed separation membranes demonstrate several major advantages over traditionally used resin-based affinity systems. Among these advantages, purification columns prepared form these materials are reusable, and do not retain any residual contaminating materials form prior fluids that the column may have been exposed to, such as contaminating BSA and other proteins.

In one embodiment, antibody capture was accomplished by injecting a sample fluid onto a purification column while running equilibration buffer (50 mM sodium phosphate pH 7.0) and eluting antibody by running a gradient of mild. elution buffer (3M NaCl in 50 mM phosphate pH 7.0). Purity of antibody yield was greater than 90%, and the efficiency for selected antibody of interest was also greater than 90% using the herein described systems and methods. For example, results using the m-NBS^{Tryptamine} column demonstrated an efficiency for selected antibodies of >98%, with a purity level of >98%.

### BRIEF DESCRIPTION OF THE DRAWINGS

FIG. 1A-FIGS. 1B—1A) Location of the nucleotide binding site (NBS) is shown on the crystal structure of the antibody Fab variable domain. 1B) Schematic representation of antibody capture with tryptamine-conjugated membrane; and

FIG. 2A-FIG. 2B—Presents chromatograms demonstrating the effect of the m-NBS^{tryptamine} column's capture efficiency. 2A) Increasing concentrations of the antibody at 10 μL 2B) Increasing volume of antibody at 0.5 mg/mL.

FIG. 3A FIG. 3C-3A) Chromatograms demonstrating the effects of changing EQ Buffer wash time on retention of Rituximab by the m-NBS^{Tryptamine} column. 3B) Control column packed with RC membranes without tryptamine modification displayed no capture of antibody or contaminants. 3C) The m-NBS^{Tryptamine} column did not display any nonspecific binding for an array of contaminants.

FIG. 4A FIG. 4C—4A) Chromatograms of Rituximab premixed with increasing BSA content. 4B) ELISA results illustrating percent antibody in the flow through and elution fractions. Data represents the means (±SD) of triplicate experiments. 4C) SDS-PAGE analysis showing no BSA contamination in recovered antibody fractions.

FIG. 5A-FIG. 5C—5A) Chromatograms of Rituximab prepared in 2 mg/mL BSA, H929 Cell Supernatant, H929 Cell Lysate, Mouse Ascites and 3T3 Cell Lysate. 5B) ELISA results illustrating percent antibody in flow through and elution fractions, Data represents the means (±SD) of triplicate experiments. 5C) SDS-PAGE analysis showing no protein contamination in recovered antibody fractions.

FIG. 6A-FIG. 6C—Presents chromatograms illustrating specificity of the m-NBS^{tryptamine} column to active, full-length antibodies. 6A) Comparison of active, denatured and 1:1 mixture of active and denatured antibodies. 6B) Mixture of active antibodies with increasing concentration of denatured antibodies. 6C) Flow cytometry results showing the binding activity of native and purified antibodies.

FIG. 7A-FIG. 7B—Effect of injection number on antibody recovery by the m-NBS^{tryptamine} column. 7A) Overlaid chromatograms of Rituximab injections (0.5 mg/mL, 10 μL) on the m-NBS^{Tryptamine} column. 7B) Percent antibody recovery based on 220 nm peak integration of the Rituximab injections. Average represents the mean (±SD) of the five Rituximab injections.

FIG. 8A FIG. 8 B—Functionalization of RC membranes with tryptamine molecule. 8A) Immobilization of tryptamine ligand on RC membrane. 8 B) Characterization of modified RC membranes by FTIR analysis.

FIG. 9 is Schematic of m-NBS^{Tryptamine} column packing into the cartage and then placing into a guard column.

FIG. 10 is Flow through fractions of impurity injections were run on a SDS-PAGE gel.

FIG. 11A FIG. 11B—11A) Chromatograms illustrating the effect of NaCl concentration in the injection buffer on antibody capture efficiency by tryptamine column. 11 B) Normalized peak integration values of the flow through and elution fractions are shown for the above injections.

FIG. 12 is Quant-iT™ PicoGreen dsDNA High Sensitivity Assay Kit standard curve. The amount of dsDNA present in the samples was determined based on dye fluorescence with a 485 nm excitation and 523 nm emission using the provided standard concentrations of dsDNA and by following the manufacturer recommended protocol. The data was fit by linear regression with R2 value of 0.998. Data represents the means (±SD) of triplicate experiments. dsDNA High Sensitivity Assay Kit standard curve.

FIG. 13 is Host cell protein (HCP) content standard curve was determined using a 3rd generation CHO HCP ELISA kit from Cygnus Technologies. The recommended high sensitivity assay as provided by the manufacturer was followed.

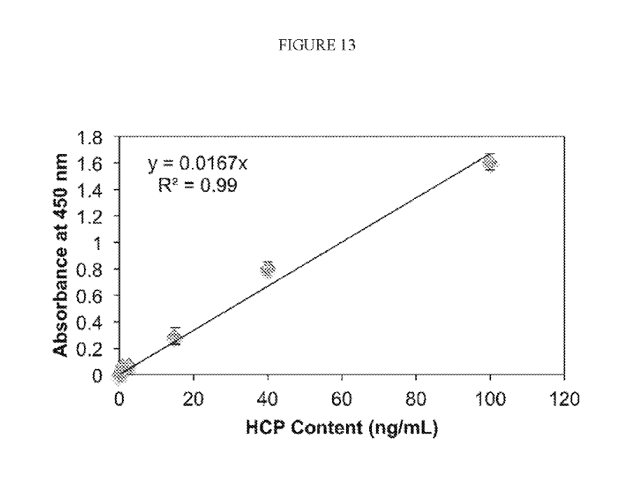

FIG. 14 is Screening of IM9 and H929 cell lines using flow through and elution fractions of purified Rituximab by m-NBS^{Tryptamine} column to access CD20 expression levels.

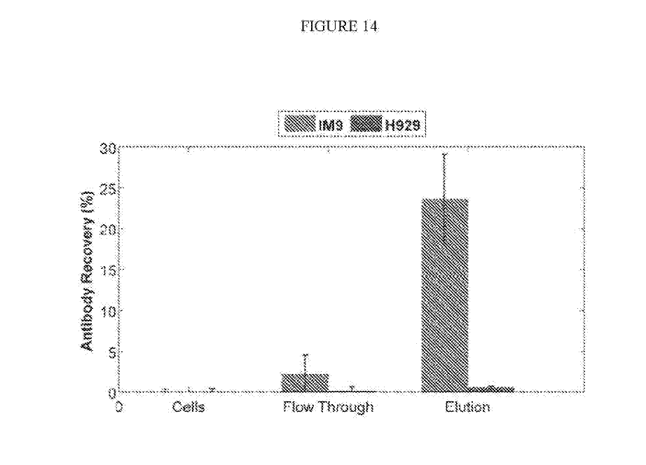

FIG. 15 is Acetone injection (30 μL) on m-NBS^{Tryptamine} column. An acetone pulse was injected onto the m-NBS^{Tryptamine} column to determine the theoretical number of plates based on peak retention time (tᵣ) and the width of the peak at 1/10 maximum peak intensity (W_{b}) plugged into the below equation to get N=107.09. The HEPT (height equivalent to the theoretical plates) value is calculated by taking the column length (1 cm) and dividing it by the calculated theoretical number of plates to get HEPT=0.019 cm. Peak asymmetry, 1.096, was determined using Agilent Technologies ChemStation LC Software.

### DETAILED DESCRIPTION OF THE PREFERRED EMBODIMENTS

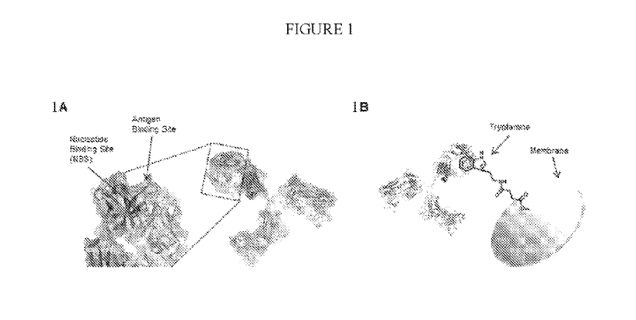

The method utilizes the nucleotide-binding site (NBS), located between heavy and light chains of an antibody (variable region of the Fab arms). This particular region is a highly conserved region in almost all antibodies (FIG. 1A) [41, 42].

The nucleotide binding site has been characterized using molecular modeling, and was found to implicate four critical residues, two tyrosine residues on the variable region of light chain (VL) (Tyr42 and Tyr103) and one tyrosine (Tyr 103) and one tryptophan (Trp118) on the variable region of heavy chain (VH) [41]. Although this region is not widely known and has no known function, it has been discovered that it has a moderate binding affinity to small hydrophobic, ring structured molecules, such as those molecules that contain an indole ring.

It has been shown that indole-3-butyric acid (IBA) has a moderate binding affinity to the highly“conserved” NBS region described here, with a Kd=1-8 μM [43]. The site-specific binding of IBA, for example, to the antibody NBS region, may be used for conjugating various peptide linkers and functionalities that contain a terminal IBA molecule to an antibody of interest. UV-photocross linking methods utilizing nucleotide binding site, UV-NBS, UV-NBS^{Biotin} and UV-NBS^{Thiol}, have been developed as universal methods for antibody [42-44] and Fab [45, 46] functionalization, as well as for use in oriented surface immobilization. These studies showed that various modifications to the NBS using intact antibodies do not affect antibody functionality, structure or antigen recognition [41, 45]. The usage of IBA as a target molecule in resin-based antibody purification systems has previously been described [33]. However, neither IBA or other materials similar to it have been used with non-resin based purification systems, and not with regenerated cellulose membrane systems.

The present methods utilize the NBS to selectively capture and purify antibodies by conjugating tryptamine to regenerated cellulose membranes to generate an NBS targeting affinity membrane column (mNBS^{Tryptamine}) (FIG. 1B).

Throughout the specification and claims, the following terms take the meanings explicitly associated herein, unless the context clearly dictates otherwise.

The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

As used herein, the term “or” is an inclusive “or” operator and is equivalent to the term “and/or” unless the context clearly dictates otherwise.

The term “based on” is not exclusive and allows for being based on additional factors not described, unless the context clearly dictates otherwise.

The term “a,” “an,” and “the” include plural references. Thus, “a” or “an” or “the” can mean one or more than one. For example, “a” cell and/or extracellular vesicle can mean one cell and/or extracellular vesicle or a plurality of cells and/or extracellular vesicles.

The meaning of “in” includes “in” and “on.”

As used herein, the terms “administering”, “introducing”, “delivering”, “placement” and “transplanting” are used interchangeably and refer to the placement of the extracellular vesicles of the technology into a subject by a method or route that results in at least partial localization of the cells and/or extracellular vesicles at a desired site. The cells and/or extracellular vesicles can be administered by any appropriate route that results in delivery to a desired location in the subject where at least a portion of the cells and/or extracellular vesicles retain their therapeutic capabilities. By way of example, a method of administration includes intravenous administration (i.v.).

As used herein, the term “treating” includes reducing or alleviating at least one adverse effect or symptom of a disease or disorder through introducing in any way a therapeutic composition of the present technology into or onto the body of a subject.

As used herein, “therapeutically effective dose” refers to an amount of a therapeutic agent (e.g., sufficient to bring about a beneficial or desired clinical effect). A dose could be administered in one or multiple administrations (e.g., 2, 3, 4, etc.). However, the precise determination of what would be considered an effective dose may be based on factors individual to each patient, including, but not limited to, the patient's age, size, type or extent of disease, stage of the disease, route of administration, the type or extent of supplemental therapy used, ongoing disease process, and type of treatment desired (e.g., cells and/or extracellular vesicles as a pharmaceutically acceptable preparation) for aggressive vs. conventional treatment.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “pharmaceutical preparation” refers to a combination of the A1 exosomes, with, as desired, a carrier, inert or active, making the composition especially suitable for diagnostic or therapeutic use in vitro, in vivo, or ex vivo.

As used herein, the terms “pharmaceutically acceptable” or “pharmacologically acceptable” refer to compositions that do not substantially produce adverse reactions, e.g., toxic, allergic, or immunological reactions, when administered to a subject. For example, normal saline is a pharmaceutically acceptable carrier solution.

As used herein, the terms “host”, “patient”, or “subject” refer to organisms to be treated by the preparations and/or methods of the present technology or to be subject to various tests provided by the technology.

The term “subject” includes animals, preferably mammals, including humans. In some embodiments, the subject is a primate. In other preferred embodiments, the subject is a human.

As used herein, the term “treating” includes reducing or alleviating at least one adverse effect or symptom of a disease or disorder through introducing in any way a therapeutic composition of the present technology into or onto the body of a subject.

As used herein, the term “effective amount” refers to the amount of a composition sufficient to effect beneficial or desired results. An effective amount can be administered in one or more administrations, applications or dosages and is not intended to be limited to a particular formulation or administration route.

The term “subject” includes animals, preferably mammals, including humans. In some embodiments, the subject is a primate. In other preferred embodiments, the subject is a human.

The following examples are provided to demonstrate and further illustrate certain preferred embodiments and aspects of the present technology, and they are not to be construed as limiting the scope of the technology.

It is specifically intended that the present invention not be limited to the embodiments and illustrations contained herein, but include modified forms of those embodiments including portions of the embodiments and combinations of elements of different embodiments as come within the scope of the following claims.

### Example 1—Materials and Methods Materials

RC 60 (Regenerated Cellulose) Membrane Filters (1.0 um, Diameter 47 mm) were purchased from Whatman™ (Germany). Tryptamine, N,N-diisopropylethylamine (DIEA), Sodium phosphate monobasic monohydrate, and mouse ascites fluid (clone NS-1) were all purchased from Sigma-Aldrich (St. Louis, Mo.). Bovine serum albumin, Fraction V was purchased from EMO Chemicals (Gibbstown, N.J.). HRP-conjugated goat anti-human lgG Fcy-specific was purchased from Jackson ImmunoResearch (West Grove, Pa.). 2-(1H-Benzotriazole-1-yl)-1,1,3,3-tetramethyluronium hexafluorophosphate (HBTU), Amicon Ultra centrifugal filters (0.5 ml, 10K), and Coomassie R-250 were purchased from EMO Millipore (Billerica, Mass.). Tris-Gly running buffer, transfer buffer, and tris buffered saline (TBS) were purchased from Boston Bioproducts (Ashland, Mass.). Amplex Red assay kit and Quant-iT PicoGreen dsDNA high-sensitivity assay kit were purchased from Invitrogen (Grand Island, N.Y.). The third-generation CHO host cell protein (HCP)enzyme-linked immunosorbent assay (ELISA) kit was purchased from Cygnus Technologies (Southport, N.C.). RPM1-1640 media was purchased from Cell-Gro (Manassas, Va.), and fetal bovine serum (FBS) was from Hyclone (Thermo Scientific, Rockford, Ill.). Guard Column Holder and Guard Cartages (0.4×2 cm) were purchased from IDEX Health and Science (Oak Harbor, W A). Rituximab was gift from Dr. Navari at the Indiana University School of Medicine in South Bend, Ind.

Membrane Functionalization

Hydroxyl groups of the RC membrane were reacted with succinic anhydride for 2 h with addition of DIEA to obtain carboxyl groups on the membrane as a functional group. Carboxylated membranes were washed with DMF and DCM, and dried with airflow. Then, carboxyl groups were activated utilizing HBTU with addition of DIEA. Excess amounts of HBTU were removed by washing with DMF and DCM from the membranes to eliminate the cross reaction of HBTU with amine group of the tryptamine molecule. The tryptamine molecule was conjugated to the membrane in DMF solution under basic conditions during an overnight incubation (FIG. S1-A). Functionalized membranes were washed with DMF and DCM at least three times in order to remove excess amounts of Tryptamine molecules, and then membranes were dried with airflow. The dried membranes were stored at RT.

Characterization of Functionalized Membranes

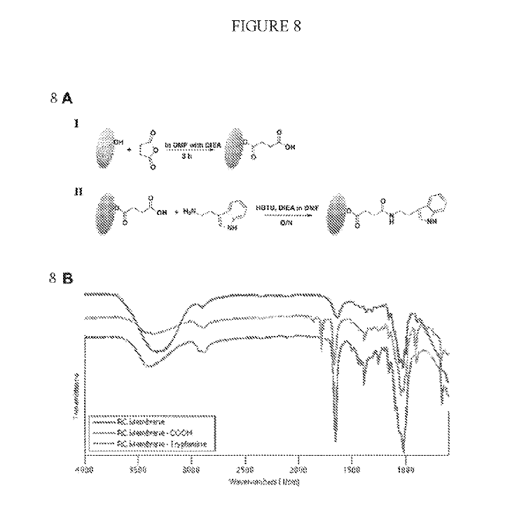

Functionalization of membranes were characterized by FTIR (FIG. 8B). Peaks at 1788 cm⁻¹ and 1662 cm⁻¹ can confirm the carboxylation of membrane. Peaks between 1400-1600 cm⁻¹ representing C-C stretches in an aromatic ring can be observed in tryptamine-functionalized membrane. Among these functional group changes, the FTIR spectrum was the same throughout the modification of RC membrane, indicating that the general membrane structure remained as stable as it initially was.

Packing Column

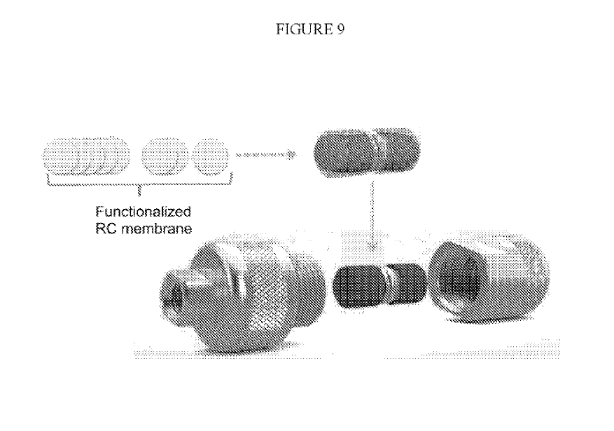

Membranes were cut into 4 mm diameter circles using a Uni-core puncher (4 mm). Post-reaction of membranes with tryptamine, all membranes were dried with airflow. Over 200 membrane circles were packed into 2 cm×4 mm cartage and then the cartage was placed into a guard column before attaching to HPLC system (FIG. 9). Packed column was equilibrated while running EQ buffer through the column for 1 h, then ELS buffer for another 1 h. In order to make sure the equilibration of the column, EQ Buffer was injected to the column and run under the same gradient condition that is used for antibody injections. This step was repeated until no change was observed on the chromatograms between the following EQ buffer injections.

Buffers and Gradient Used for Affinity Separation

An Agilent Technologies 1200 Series HPLC system was used in all chromatographic injections. Elution from affinity membrane beds predominantly employs gradient, or step changes, in eluent composition to selectively elute products. 50 mM phosphate buffer at pH 7.0 was used as an equilibration buffer (EQ) and 3 M NaCl in 50 mM phosphate buffer at pH 7.0 was used as an Elution Buffer (ELS). Unless otherwise noted following the injection of sample, the column was washed for three minutes with an EQ buffer to capture the antibody and washed away the contaminants, the antibody was then eluted using a 10-minute linear gradient from 0 to 100% ELS buffer. The column was cleaned with ELS buffer for two minutes and re-equilibrated for five minutes with the EQ Buffer.

Determination of Antibody Recovery by ELISA

The flow through and elution fractions collected from the m-NBS^{Tryptamine} column were diluted 100-fold in a 0.05 M carbonate-bicarbonate buffer pH 9.6 to a final volume of 100 μL and directly adsorbed on a high-binding Costar 96 well plate for 1.5 h. at room temperature. The surface was subsequently blocked with 2.5 g of BSA in 50 ml of phosphate-buffered saline (PBS) pH 7.4 and 0.05% Tween20 for 45 min. Total antibody in each well was determined using an HRP-conjugated secondary antibody and was quantifies using an Amplex Red assay kit (570 nm excitation and 592 nm emission).

Determination of Antibody Purity by SDS-Page

The purity of antibody in the elution fractions was determined by SDS-PAGE under reducing conditions, using 10% polyacrylamide gel with Tris-Glycine running buffer. Sample preparation was done by adding 5 μL of gel loading buffer to 15 μL of concentrated flow through or elution fraction and boiling for 5 mM. Gels were Coomassie blue stained using Coomassie R-250. The purity of the product was calculated as the fraction of the total area and intensity equivalent to the IgG bands at 25 kDa and 50 kDa. The antibody purity was determined by densitometric analysis of Coomassie-stained gels using ImageJ software.

Influence of Impurities on Antibody Recovery and Purity

The effect of impurities such as BSA, cell culture supernatant, cell lysate, and mouse ascites on m-NBS^{Tryptamine} affinity column were tested by mixing them with antibody sample in various concentration and analyzing the chromatogram. To analyze the effect of BSA on the column's performance, samples containing 0.5 mg/ml rituximab in increasing concentrations of BSA (0, 0.5, 1, 1.5, 2, 3, 5, 10, 15, 20 mg/ml) were prepared in 50 mM sodium phosphate buffer at pH 7.0.

Determination of Binding Activity of Purified Antibody

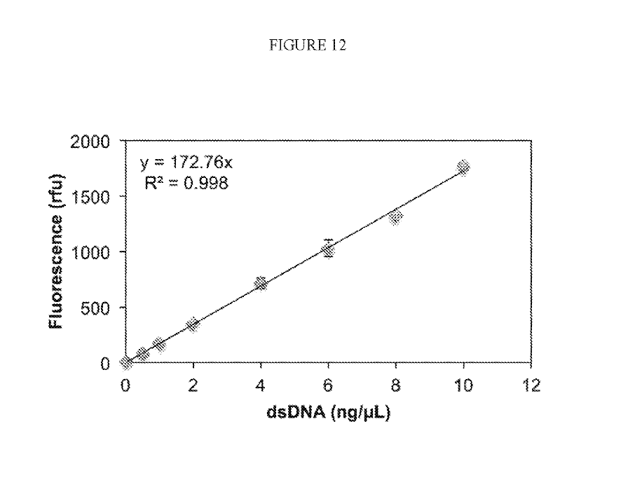

Binding activity of purified antibodies using M-NBS^{Tryptamine} affinity column were determined by flow cytometry experiments. For CD-20 expression assays, cells were incubated with Rituximab in binding buffer (1.5% BSA in PBS pH 7.4) on ice for 1 h and washed twice. IM9 cells expressing CD-20 receptor was identified for use in the present study, and this receptor is available for Rituximab binding (FIG. 12). Thus, binding activity of purified Rituximab was tested on IM9 cells. Briefly, 5×10⁵ cells were incubated into each well. After 24 h incubation, cells were washed using PBS, and blocked with 1.5% BSA in PBS for 30 min Rituximab was incubated with cells on ice for 1 h, then fluorescein conjugated anti-human lgG antibody was used to detect bound Rituximab antibodies on ice. Samples were washed twice and analyzed on a Guava easyCyte 8HT flow cytometer (Millipore).

Specificity of Affinity Chromatography to Active Antibodies

Rituximab was denatured using 4 M guanidine hydrochloride (GndCI) and by storing the antibody at room temperature for three hours. Different ratios of denatured and native antibody was mixed and injected into the m-NBS^{Tryptamine} column. Flow through (0.5-3 min) and elution peaks' areas of the chromatograms were calculated and compared.

Residual Host Cell DNA Content

The double-stranded DNA (dsDNA) content within the flow through and elution fractions was quantified via a Quant-iT PicoGreen dsDNA high sensitivity assay kit. Cell culture supernatants and mouse ascites fluid were injected on the column using the standard purification gradient. 20 μL of each collected fraction was added to 200 μL of diluted Quant-iT PicoGreen dye reagent (1:200 dilutions in the provided buffer). The solutions were mixed and allowed to incubate 5 min at room temperature in a 96 well plate protected from light. The amount of dsDNA present in the samples was determined based on dye fluorescence with a 485 nm excitation and 523 nm emission. This fluorescence was converted to nanograms per microliter of dsDNA based on a standard curve. Data represents the means (±SD) of triplicate experiments.

Residual Host Cell Protein Content

A third generation CHO HCP ELISA kit from Cygnus Technologies was used to quantify the HCP (host cell protein) content present in the flow through and elution collected fractions post m-NBSTryptamine column purification. The recommended high-sensitivity assay protocol as provided by the manufacturer was followed. Briefly, 100 μL of anti-CHO:HRP matrix was added to each well followed by 50 μL of standards, controls, and samples. The plate was covered and incubated on a rotator at room temperature for 2 h. Following incubation the plate was washed with four cycles of 350 μL of wash solution. 100 μL of 3,3′,5,5′ tetramethyl benzidine (TMB) substrate was then added to the wells and incubated for 30 min without rotating. An amount of 100 μL of stop solution was added to stop the enzymatic reaction. The amount of residual host cell protein content in the samples quantified by reading the absorbance at 450 nm subtracting off the zero standard as a blank. Data represents the means (±SD) of triplicate experiments.

### Example 2—Selection of Membrane and Preparation of Stationary Phase

The preparation of a membrane for antibody purification purposes requires several steps: i) selection of a suitable membrane, ii) activation of the membrane and then iii) immobilization of an appropriate ligand for the target molecule on the membrane [47, 48]. There are several kinds of commercially available microporous membranes that have been used for antibody purification systems with regenerated cellulose (RC). Polyethersulfone and polyvinylidene fluoride [37] are among the more common regenerated cellulose materials that have been reported.

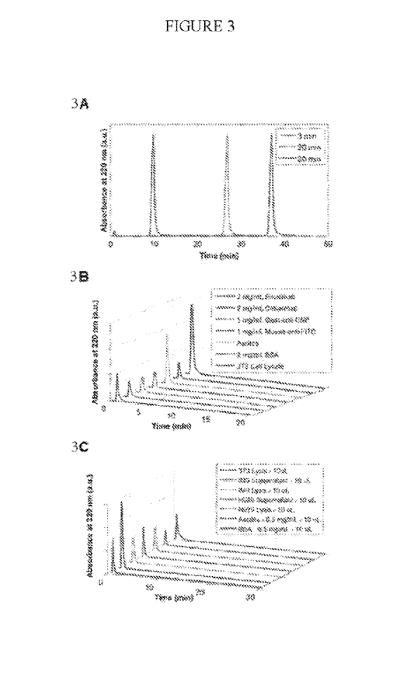

Regenerated cellulose (RC) was selected as a membrane material in the present studies. In part, this selection is due to its specific features such as its strength while wet, extreme chemical resistance and high mechanical stability. One other advantage of RC membranes is their ability to be sterilized by all methods. This is an important feature, as native and derivatized cellulose membranes are soluble only in some strong acids [62]. The hydrophilic property of RC membrane is also an advantage in antibody purification system due to the low hydrophobic interaction ability of the membrane, which eliminates non-specific interactions between the membrane and antibodies or other ingredients (FIG. 3-B). A 1 μm pore sized membrane was used in order to achieve high flow rates while keeping the pressure low.

The activation of these membranes utilizes the its hydroxyl groups as a functional group. Succinic anhydride was selected for use, as it reacts with hydroxyl groups to obtain carboxyl groups on the membrane as a functional group (FIG. 8A). Then, carboxyl groups were activated utilizing HBTU with the addition of DIEA. The tryptamine molecule was conjugated to the membrane in DMF solution under basic conditions (FIG. 1-B. Functionalization of membranes was characterized by FTIR (FIG. 8B). Peaks at 1788 cm- and 1662 cm-1 can confirm the carboxylation of membrane. Peaks between 1400-1600 cm-1 representing C-C stretches in an aromatic ring can be observed in tryptamine functionalized membrane. Among these functional group changes, the FTIR spectrum was the same throughout the modification of RC membrane, indicating that the general membrane structure remained as stable as it initially was.

The major limitation of membrane chromatography was the restriction of the flow-rate by the ligand-protein association kinetics. To overcome this potential limitation, stacks of several thin membranes were used. The columns (4 mm×2 cm) were packed with nearly 235 tryptamine-functionalized membranes to obtain an m-NBS^{Tryptamine} column. This column was then attached to an HPLC system (Agilent Technologies 1200 Series).

### Example 3—Capturing and Purification of Antibodies Via m-NBS^{Tryptamine} Column

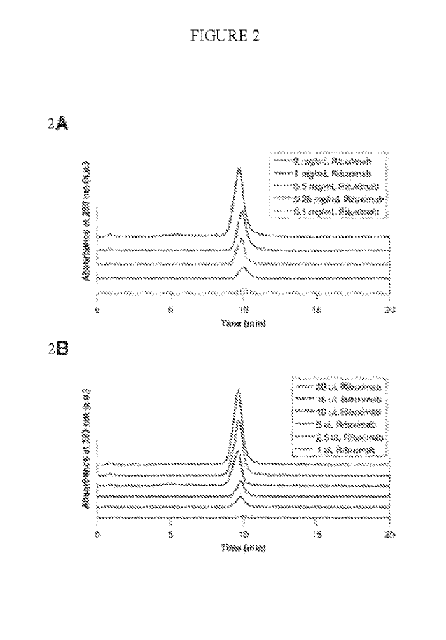

To evaluate antibody capture efficiency of the m-NBS^{Tryptamine} affinity column, Rituximab, a chimeric anti-CD20 pharmaceutical antibody, was used. The indicated amount of antibody in the EQ buffer was injected into the column, successfully captured on the column using the EQ Buffer and was then eluted using a gradient of ELS buffer. Antibody recovery was quantified by peak integration. The column-loading limit tested both the increase of the concentration of the antibody in 10 μL (FIG. 2-A) and the increase volume of antibody sample while keeping the concentration at 0.5 mg/ml (FIG. 2-B). Increasing the amount of antibody injected into the column did not effect on capture efficiency and consistently yielded>98% antibody recovery. The largest amount of antibody injected on the column was 20 μg of Rituximab (10 μL of 2 mg/ml) into a column volume of 250 μL without any sign of exceeding the column's antibody capture capacity.

### Example 4

To demonstrate that the antibody capture observed with the tryptamine modified membrane column is attributed to the affinity of tryptamine molecules to the antibody and was not due to size exclusion phenomenon, the antibody capturing and elution properties of the column at various wash times of 3, 20 and 30 minutes was tested. The antibody was retained on the column throughout the EQ wash under all conditions, and was eluted consistently for 7 minutes into the ELS gradient, leading to elution times of 10, 27 and 37 minutes, respectively (FIG. 3-A). Since the elution time is dependent on the duration of wash time, the retention of the antibody on the column is not due to a size exclusion effect of the membrane column, and the elution time would have been independent of the duration of the wash time.

To test that an inherent property of RC membrane was not the cause of antibody capturing, a control column was packed with RC membranes without a small molecule (tryptamine) modification. Various contaminants (Ascites, BSA and 3T3 Cell Lysates) and several monoclonal antibodies (Rituximab, Cetuximab, goat-anti-DNP and mouse-anti-FITC) were injected to the non-modified membrane packed column. Neither antibodies nor impurities were captured in the control column, all injected samples eluted in the flow through (0.5-3 minutes) (FIG. 3-B).

### Example 5—Specificity of m-NBS^{Tryptamine} Affinity Chromatography Column

To assess the specificity of m-NBS^{Tryptamine} column in antibody capture, various contaminants were injected to a column in order to demonstrate that the contaminants were not captured on the column. None of the proteins or other biological molecules were retained on the column, and all of the impurities eluted within the flow through (0.5-3 min) post injection (FIG. 3-C).

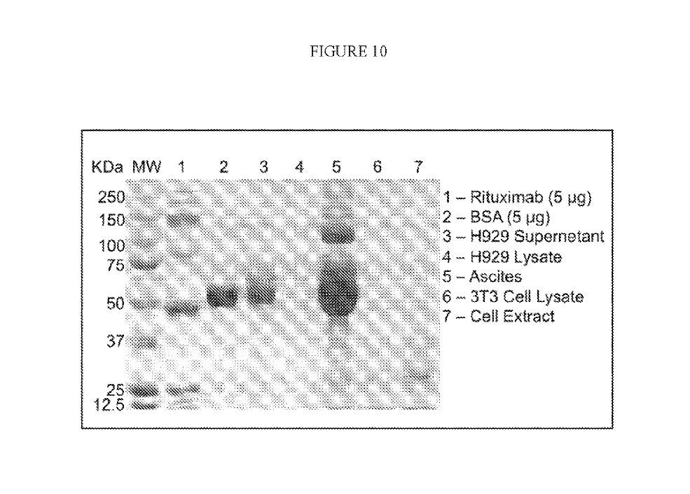

The results indicate that the m-NBS^{Tryptamine} column has a high selectivity for only antibodies with no cross-selectivity for other proteins from culture conditions. Some of flow through fraction of contaminant injections were collected and run on a SDS-PAGE gel for further characterization (FIG. 10). Additionally, efficient capture of Rituximab post-exposure to the diverse contaminants was accomplished, indicating that these contaminants do not have a residual negative impact on the affinity of the small molecule (tryptamine) column to antibodies.

Taken together, these results show that antibody capture on a non-resin affinity column having a small molecule attached thereto (such as in an m-NBS^{Tryptamine} column) is a result of specific interactions between the immobilized tryptamine on the membrane and the antibody.

In order to show the ability of a tryptamine modified membrane based chromatography column to separate antibodies from challenging impurities, antibody samples contaminated with known amounts of BSA at various concentrations were tested. Since BSA is the major impurity in the cell culture supernatants and ascites fluid, and it is also known to aggressively adhere to the antibody surface through non-specific interactions, BSA was selected as a major test criterion.

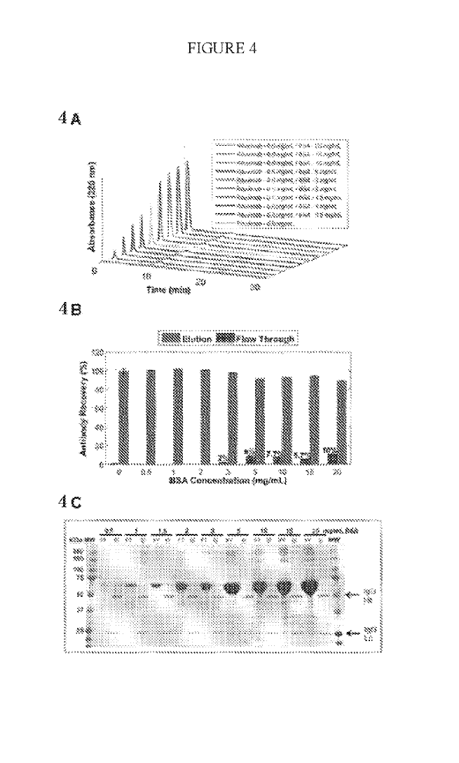

BSA contaminated antibody samples (0.5 mg/mL) were injected on a column as described above (FIG. 4A), and the flow through and elution fractions were collected for further analysis. A significant increase in the amount of BSA was detectable in the flow through fractions as the BSA contaminant amount in the injection sample increased. Nonetheless, no BSA was detectable in the antibody elution fractions, even at the highest BSA concentration, indicating that the recovery antibody fractions did not have any BSA impurity.

ELISA and SD S-PAGE were used for analyzing the purity of the antibody. On the basis of ELISA results, no significant changes were observed in the amount of antibody in the elution fractions with increasing BSA concentrations (FIG. 4B). Some levels of antibody, however, were detectable in the flow through of the BSA-contaminated fractions that ranged from 2 to 10% of the total amount of injected. These results suggest that contaminating the antibody samples with BSA resulted in only a slight reduction in antibody recovery at higher BSA concentrations; according to the ELISA, 90-98% antibody recovered compared to >98% in the contaminant-free injection. It is noteworthy that the m-NBS^{Tryptamine} column performed adequately even at the highest BSA concentration used in these experiments, although such extreme conditions are not representative of the biological fluids antibodies are typically isolated from. (normal albumin range in serum is 3.5-4.7 g/dL [49].)

The purity of the antibody was analyzed by SDS-PAGE analysis (FIG. 4C). A significant increase in the amount of BSA was detectable in the flow through as the BSA contaminant amount in the injection sample increased. Nevertheless, no BSA was detectable in the elution fractions even at the highest BSA concentration, indicating that the recovered antibody fractions did not have any BSA impurity.

### Example 6 Column Efficiency to Purify Antibody from Other Typical Contaminant Sources Along with BSA: Conditioned Cell Culture Supernatants, Cell Lysates and Ascites Fluid

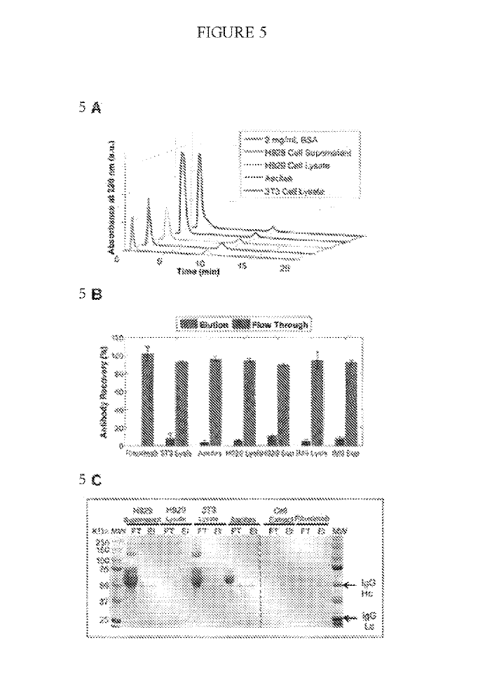

Samples of 0.5 mg/mL antibody were mixed with these contaminants and subsequently purified utilizing tryptamine column (FIG. 5-A). The flow through and elution peaks were collected and analyzed for antibody recovery and purity using ELISA (FIG. 5-B) and SDS-PAGE (FIG. 5C). ELISA results indicate no significant loss in antibody recovery from any of the tested contaminant sources. According to the SDS-PAGE results, all contaminants eluted within the flow through fraction.

None of the impurities were detectable within the elution fraction, indicating that tryptamine column performed adequately to purify antibodies from biological environment. Combined, the tryptamine column achieved successfully separate proteins and other contaminant from the culture media and purify the antibody with the yield of >95%.

### Example 7—Removing of Host Cell Proteins and Host Cell DNA

Therapeutic antibodies are most commonly produced in cell culture processes. As a consequence, the cells from the culture media are the largest source of contaminants, which include host cell proteins (HCPs) and DNA. Therefore, host cell DNA removal from the purified antibody was determined via binding of fluorescent dye to dsDNA present in the flow through and elution fractions of the antibody purified from various contaminant sources including conditioned cell culture supernatant, lysates and ascites. This fluorescence was converted to nanograms per microliter of dsDNA by using a standard curve (FIG. 12) and then normalized to antibody content in each fraction.

Table 1 shows a summary of DNA content in the collected flow through and elution fractions with log reduction value (LRV). LRV was calculated by taking the logarithm of the ratio of load (sum of flow through and elution) to elution fractions. The results demonstrate that DNA flows through the column relatively unimpeded by the tryptamine or membrane leaving a very low level of DNA in the purified antibody elution fraction with a of >2, running congruently with protein A DNA clearance values [23, 50].

TABLE 1

| | Flow Through | Elution DNA | |
|---|---|---|---|
| Sample | DNA (ng/mg mAb) | (ng/mg mAb) | LRV |
| 3T3 | 283779578.6 | 217129.9 | 3.11 |
| Cell Extract | 874710812.7 | 92303.5 | 3.98 |
| Ascites | 116240796.5 | 116361.6 | 2.99 |
| H929 Lysates | 17301227.1 | 71931.1 | 2.38 |
| H929 Supernatant | 140062977.5 | 47670.6 | 3.47 |
| IM9 Lysates | 10555221.12 | 69946.54 | 2.18 |
| IM9 Supernatant | 170364436.2 | 192666.9 | 2.95 |

Furthermore, residual HCP content in each collected fraction was analyzed via a broadly reactive HCP ELISA assay by using standard curve (FIG. S6). The concentration of HCP present in the initial rituximab/contaminant source solution (sum of flow through and elution fractions) was compared to the purified product. The LRV values were >7 and around 2 when Rituximab was purified from 3T3 cell conditioned media and from other impurities, respectively. These LRV values are comparable to that of protein A chromatography [23, 51]. A summary of the HCP content in the collected fractions is shown in Table 2. These results further support the high level of purity (>98%) that the tryptamine column technique can attain.

TABLE 2

| | Flow Through | Elution HCP | |
|---|---|---|---|
| Sample | HCP (ng/mg mAb) | (ng/mg mAb) | LRV |
| 3T3 | 13493317.4 | 0.2 | 7.82 |
| Cell Extract | 817437.1 | 5618.6 | 2.17 |
| Ascites | 1063185.6 | 2715 | 2.59 |
| H929 Lysates | 2538970.1 | 4185.7 | 2.78 |
| H929 Supernatant | 1139497 | 1923.2 | 2.77 |
| IM9 Lysates | 237988 | 8145.1 | 1.48 |
| IM9 Supernatant | 560047.9 | 1432.9 | 2.59 |

### Example 8—Effectively Eluting Captured Antibodies from a Column

To evaluate the efficiency of antibody capture by tryptamine column depending on the ionic strength of the sample injection buffer, various concentration of NaCl in the sample injection buffer are examined in the present example.

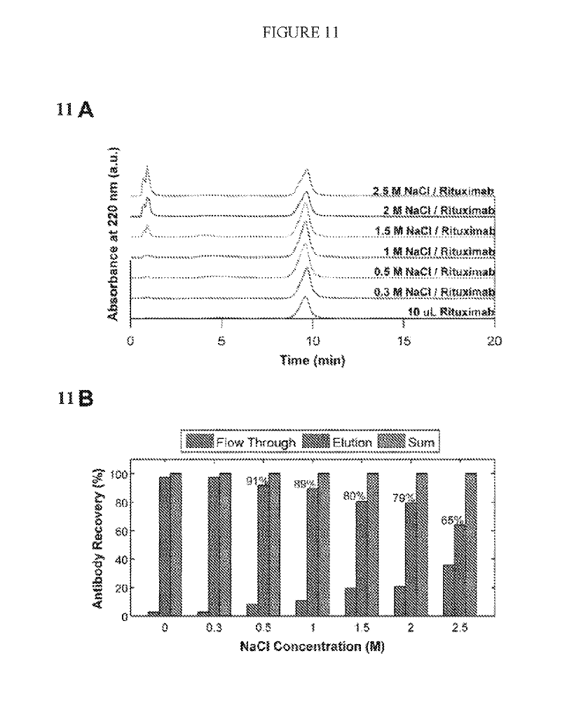

In this study, antibody samples were prepared in EQ buffer with increasing NaCl concentrations and injected them into the column (FIG. 11A). All injections were run under the same purification gradient and buffers as the previous ones in order to be able to purely and accurately test the ionic strength of the sample on the column. Antibody recovery was quantified by comparing the peak integration values, at 220 nm, of the flow through and elution fractions (FIG. 11B).

The integration values from each peak were summed, and continued that the entire injected antibody sample eluted from the column, and also that addition of NaCl did not promote irreversible antibody binding to the column. Since high salt concentration of the ELS buffer drives antibody elution, it was possible that there would be a decreased amount of tryptamine column capture efficiency with increasing concentrations of NaCl. At the highest concentrations of NaCl examined (2.5 M), a recovery rate of 65% was still maintained employing the present techniques, compared to an about 80% recovery rate at 1.5 M NaCl and 2.0 M NaCl. A recovery rate of about 90% was observed with both 0.5 M NaCl and 0.3 M NaCl (FIG. 11B).

### Example 9—Retaining Binding Activity of Purified Antibodies after Purified Via m-NBS^{Tryptamine} Column

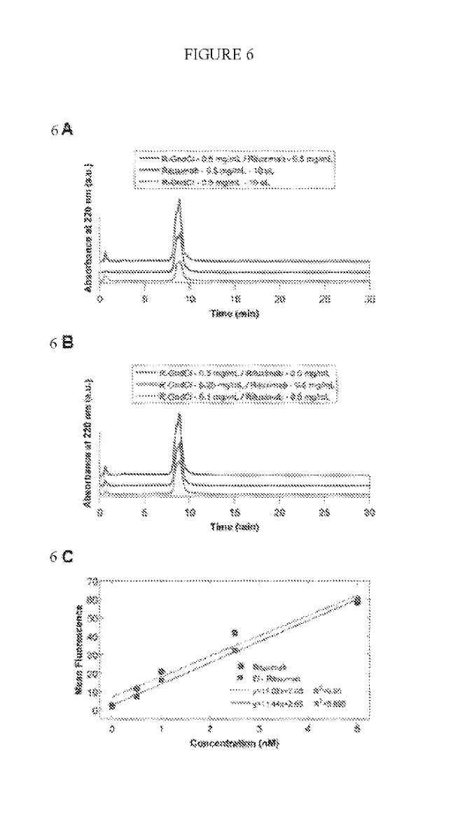

To demonstrate that the affinity-based chromatography method is specific for active antibodies, we purified the active antibody from a solution containing both denatured and active antibody. Rituximab was chemically denatured using 4 M GndCl, incubating at room temperature for three hours. Then, the buffer of the denatured antibodies including 4 M GndCl was changed to PBS in order to stop the reaction and eliminate further denaturing event post mixing with active antibodies. 10 μL of denatured antibodies, active antibodies and 1:1 mixture of denatured and active antibodies were injected into the column, and both integration of flow through and elution were compared (FIG. 6A). The sum of flow through fractions integrated area of denatured antibody (205.5) and active antibody (31.5) were perfectly matched with integrated flow through area of mixture sample (234.5). A similar trend was also observed with elution fractions. The elution fraction area of denatured antibody (1589.1) and active antibody (3223) sum (4812.1) give similar integrated area of mixture sample (4378). Furthermore, to show specificity of the column to active antibody, increasing concentrations of denatured antibody was mixed with same amount of active antibody, and we were able to elute increasing peaks of flow through fractions with increasing amount of the denatured antibody (FIG. 6-B). These results demonstrate specificity of the tryptamine column for active antibody from a solution containing a mixture of active and damaged or denatured antibody.

### Example 10—Purification of Active Rituximab from a Solution of Active and Denatured Antibody

The binding activity of the purified antibodies is examined in the present example.

Binding activity of the purified antibodies was accomplished through the analyzation of binding of antibodies to cell lines that expressed specific target proteins. Rituximab (antibody) targets human CD20 and was evaluated using the CD20 expressing multiple myeloma cell line IM-9 [41, 52]. Both native and purified Rituximab was incubated with IM-9 cell lines at increasing concentrations for 3 hours on ice. Binding was detected using a secondary Fc specific fluorescein labeled antibody. The slope of the mean fluorescence curve was used to determine the binding activity. Purified antibody shows similar binding activity (slope: 11.44 R²=0.996) with comparison to native antibody (slope: 11.02 R²=0.95) (FIG. 6C).

These results demonstrated that the antibodies that were purified through the tryptamine column had native levels of binding to the cells. This further validated the tryptamine column purification and confirmed that the antibody activity (including both antigen detection and Fc recognition) was not adversely affected post purification.

### Example 11—Column Stability and Reusability

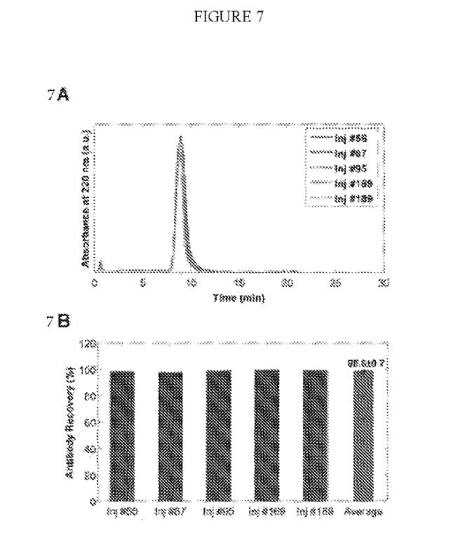

In this example, nearly 200 injections of rituximab antibody were performed into m-NBS^{Tryptamine} column. Results of rituximab injections without mixing with any contaminants are shown in FIG. 7A.

On the basis of elution time, peak elution profile, and peak intensity there was no discernable modification to the capture ligand. The m-NBS^{Tryptamine} affinity column yielded reproducible results without a loss in performance in antibody recovery (98.5±0.7) even after 189 injections (FIG. 7B). Within these 200 cycles of injections, the column was taken off from the HPLC system and attached again after several weeks, which did not result any reduction in the column stability or performance.

To confirm the quality and consistency of the chromatographic operations, the integrity of the column bed was measured. For this purpose, an acetone pulse was injected on the tryptamine column to determine the theoretical number of plates based on peak retention time (tᵣ) and the width of the peak at 1/10 maximum peak intensity (W_{b}) plugged into the Equation 1 to get N=107.09.

$$N = 16 ( \frac{{t}^{r}}{{W}^{b }} ) 2 \qquad Eq . 1$$

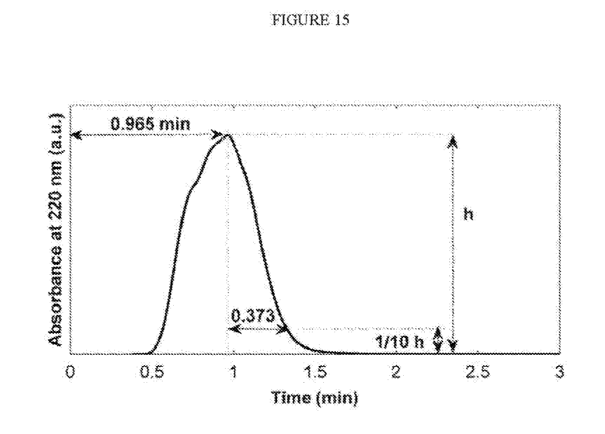

For an appropriate pulse test, it is necessary to inject a sufficiently large volume of organic solvent so that it remains undiluted by the hold-up volumes. Generally, it is suggested that injection volumes is 1-2.5% of the column volumes. Therefore, 30 μL of acetone (injection volume) was used for 250 μL hold-up volume column, which results 12% (FIG. 15).

The HETP (height equivalent to the theoretical plates) value was calculated by taking the column length (2 cm) and dividing it by the calculated theoretical number of plates to get HETP=0.019 cm. HETP≤0.02 cm represents good packing of column. Another good packing criteria, peak asymmetry, 1.096, was determined using Agilent Technologies ChemStation LC software, and found within the acceptable range between 0.8 and 1.4.

Small molecule targeted chromatography systems, with their durability and long-term usage capability, as described herein, provide high efficiency and extended life use as part of an antibody purification system. Usage of small molecules in purification systems, however, present somewhat of a problem associated with a limited antibody capturing efficiency. This study demonstrates an optimized affinity membrane chromatography method utilizing the NBS for selective purification of antibodies from complex media. This small molecule targeted affinity chromatography method provide>98% antibody recovery with >98% purity during purifications that are perforated with various contaminants such as BSA, conditioned cell culture media, and ascites fluid. m-NBS^{Tryptamine} affinity column yielded highly selective antibody purification profile to bivalently active intact antibodies.

The present methods demonstrate an antibody purification technique with a reusable column that provides consistently reproducible results without a significant loss in performance in antibody recovery, even after nearly 200 injections (runs/uses).

The membrane—nucleotide binding site (m-NBS) affinity column, for example, the m-NBS^{Tryptamine}) affinity column, provides a superior methodology for purification of antibodies, particularly humanized and chimeric antibodies. These methods provide several advantages over other techniques, such as those that employ a protein-A affinity purification method. Among other advantages, the present methodologies present a more economical approach for producing higher volumes of purified antibodies, thus increasing the affordability and availability of antibody based treatment and diagnostic systems to patients.

### BIBLIOGRAPHY

1. E. Lobo, R. Hansen, J. Balthasar, J. Pharm. Sci. 93 (2004) 2645-2668.
2. B. Lu, M. R. Smyth, R. O'Kennedy, Analyst 121 (1996) 29R-32R.
3. J. J. Gray, Curr. Opin. Struct. Biol. 14 (2004) 110-115.
4. A. Makaraviciute, et al., Biosensors and Bioelectronics 50 (2013) 460-471.
5. N. S. Ferreira, M. G. F. Sales, Biosensors and Bioelectronics 53 (2014) 193-199.
6. W. Zhang, et al., ACS nano 8 (2014) 1419-1428.
7. A. Beck, et al., Nature Reviews Immunology 10 (2010) 345-352.
8. C. Emmons, L. G. Hunsicker, Iowa Med. 77 (1987) 78-82.
9. G. G. Bornstein, The AAPS journal (2015) 1-10.
10. A. L. Nelson, et al., Nature reviews drug discovery 9 (2010) 767-774.
11. U. Gottschalk, Biotechnol. Prog. 24 (2008) 496-503.
12. D. Low, R. O'Leary, N. S. Pujar, Journal of Chromatography B 848 (2007) 48-63.
13. J. Thommes, M. Kula, Biotechnol. Prog. 11 (1995) 357-367.
14. S. Hober, K. Nord, M. Linhult, Journal of Chromatography B 848 (2007) 40-47.
15. A. C. A. Roque, C. S. O. Silva, M. A. Taipa, J. Chromatogr. A 1160 (2007) 44-55.
16. A. C. A. Roque, C. R. Lowe, M. A Taipa, Biotechnol. Prog. 20 (2004) 639-654.
17. H. Zou, Q. Luo, D. Zhou, J. Biochem. Biophys. Methods 49 (2001) 199-240.
18. A. A. Shukla, et al., J. Chromatogr. B 848 (2007) 28-39.
19. B. Kelley, Biotechnol. Prog. 23 (2007) 995-1008.
20. J. A. C. Lim, A. Sinclair, D. S. Kim, U. Gottschalk, BioProcess Int 5 (2007) 60-64.
21. T. Lakvist, et al., Acta Pathologica Microbiologica Scandinavica 56 (1962) 295-304.
22. H. Hjelm, K. Hjelm, J. Sjoquist, FEBS Lett. 28 (1972) 73-76.
23. A. D. Naik, et al., J. Chromatogr. A 1218 (2011) 1691-1700.
24. G. Fassina, et al., Biochem. Biophys. Methods 49 (2001) 481-490.
25. C. Jiang, J. Liu, et al. J. Chromatogr. A 1216 (2009) 5849-5855.
26. H. Feng, L. Jia, H. Li, X. Wang, Biomed. Chromatogr. 20 (2006) 1109-1115.
27. R. Hahn, et al., J. Chromatogr. A 1102 (2006) 224-231.
28. A. Verdoliva, et al., Chembiochem 6 (2005) 1242-1253.
29. P. Calmettes, L. Cser, E. Rajnavolgyi, Arch. Biochem. Biophys. 291 (1991) 277-283.
30. T. Arakawa, et al., Biochimica et Biophysica Acta (BBA)-Proteins and Proteomics 1844 (2014) 2032-2040.
31. T. Arakawa, et al., Protein Expr. Purif. 36 (2004) 244-248.
32. J. M. Lee, et al., Anal. Chem. 79 (2007) 2680-2687.
33. N. J. Alves, et al., Anal. Chem. 84(18) (2012) 7721-8.
34. Y. Wei, J. Xu, L. Zhang, Y. Fu, X. Xu, RSC Advances, 5 (2015) 67093-67101.
35. Q. Luo, H. Zou, Q. Zhang, X. Xiao, J. Ni, Biotechnol. Bioeng. 80 (2002) 481-489.
36. T. C. Bog-Hansen, Mol. Biotechnol. 8 (1997) 279-281.
37. S. Govender, et al., Journal of Chromatography B 859 (2007) 1-8.
38. R. Ghosh, Journal of Chromatography A 952 (2002) 13-27.
39. T. Warner, S. Nochumson, Pharmaceutical Technology, September (2002).
40. V. Orr, L. Zhong, M. Moo-Young, C. P. Chou, Biotechnol. Adv. 31(2013) 450-465.
41. N. J. Alves, et al., Biomaterials 34 (2013) 5700-5710.
42. N. J. Alves, N. Mustafaoglu, B. Bilgicer, Biosens. Bioelectron. 49 (2013) 387-393.
43. N. J. Alves, T. Kiziltepe, B. Bilgicer, Langmuir 28 (2012) 9640-9648.
44. N. J. Alves, N. Mustafaoglu, B. Bilgicer, Bioconjug. Chem. 25 (2014) 6534-6541.
45. N. Mustafaoglu, N. J. Alves, B. Bilgicer, Langmuir 31 (2015) 9728-9736.
46. N. Mustafaoglu, N. J. Alves, B. Bilgicer, Biotechnol. Bioeng. 112 (2015) 1327-1334.
47. Z. Feng, Z. Shao, J. Yao, Y. Huang, X. Chen, Polymer 50 (2009) 1257-1263.
48. C. Boi, Journal of Chromatography B 848 (2007) 19-27.
49. T. B. Horwich, et al., Am. Heart J. 155 (2008) 883-889.
50. M. D. Butler, B. Kluck, T. Bentley, J. Chromatogr. A 1216 (2009) 6938-6945.
51. A. A. Shukla, P. Hinckley, Biotechnol. Prog. 24 (2008) 1115-1121.
52. J. F. Stefanick, J. D. Ashley, B. Bilgicer, ACS Nano 7 (9) (2013) 8115-8127.

We claim:

1. A method for purifying an antibody of interest comprising:
providing a separation column comprising a separation matrix, said separation matrix having affixed thereto a small molecule capture ligand having binding affinity for a nucleotide binding site (NBS) of an antibody of interest;
providing a sample from which the antibody of interest will be purified to the separation matrix, wherein said small molecule capture ligand will bind antibody of interest present in the sample; and
eluting the separation column with an elution fluid, wherein elution fractions corresponding to fractions containing the antibody of interest fractions are collected; and
purifying the antibody of interest from the collected fractions.

2. The method of claim 1 wherein the separation matrix is a regenerated cellulose membrane.

3. The method of claim 2 wherein the regenerated cellulose membrane comprises polyethersulfone or polyvinylidene fluoride.

4. The method of claim 1 wherein said antibody of interest is a monoclonal antibody or polyclonal antibody

5. The method of claim 1 wherein said antibody is a native antibody or a recombinant antibody.

6. The method of claim 5 wherein the recombinant antibody is a chimeric antibody.

7. The method of claim 6 wherein the chimeric antibody is a humanized monoclonal antibody.

8. The method of claim 1 wherein the antibody is Rituximab

9. The method of claim 1 wherein the small molecule capture ligand is a sequence having binding affinity for the NBS of the antibody of interest and an indole ring structure.

10. The method of claim 9 wherein said small molecule capture ligand is tryptamine.

11. The method of claim 9 wherein the NBS comprises a sequence of a variable domain region of an FAB region of the antibody of interest.

12. The method of claim 1 wherein the fluid is a cell culture media in which cells have been cultured or a biological fluid.

13. The method of claim 12 wherein the sample is a biological fluid or residual biological fluid.

14. The method of claim 13 wherein the biological fluid is ascites fluid, blood, serum, or plasma.

15. The method of claim 1 wherein the said antibody is a therapeutic antibody.

16. The method of claim 15 wherein the therapeutic antibody is an anti-cancer therapeutic agent.

17. A reusable antibody purification synthetic substrate comprising:
a solid substrate comprising a synthetic separation matrix;
a small molecule affinity ligand conjugated to said substrate, wherein said small molecule affinity ligand has an indole structure and demonstrates binding affinity for a nucleotide binding site (NBS) of a mammalian antibody,
wherein said synthetic separation matrix is functionalized to include carboxyl groups to provide a carboxylated membrane.

18. The reusable antibody purification synthetic substrate of claim 17 wherein the small molecule affinity ligand is tryptamine.

19. The reusable synthetic substrate of claim 17 wherein the synthetic separation matrix comprises a regenerated cellulose membrane.

20. The method of claim 19 wherein the regenerated cellulose membrane comprises polyethersulfone or polyvinylidene fluoride.

21. An antibody purification kit comprising an antibody purification column, said antibody purification column comprising the reusable antibody purification synthetic substrate of claim 17 and an instructive insert.
